(19) United States
(12) Patent Application Publication
(10) Pub. No.: US 2014/0128333 A1
(43) Pub. Date: May 8, 2014

(54) Compounds, Compositions, and Methods for Decreasing Intestinal Glucose Uptake and Inducing Incretin Release

(71) Applicants: Juan Luis HANCKE OROZCO, Santiago (CL); Rafael Agustin BURGOS AGUILERA, Valdivia (CL); Evelyn Liliana JARA FERNANDEZ, Valdivia (CL)

(72) Inventors: Juan Luis HANCKE OROZCO, Santiago (CL); Rafael Agustin BURGOS AGUILERA, Valdivia (CL); Evelyn Liliana JARA FERNANDEZ, Valdivia (CL)

(73) Assignee: MAQUI NEW LIFE S.A., Santiago (CL)

(21) Appl. No.: 14/067,066

(22) Filed: Oct. 30, 2013

Related U.S. Application Data
(60) Provisional application No. 61/721,738, filed on Nov. 2, 2012.

(51) Int. Cl. A61K 31/7048

(52) U.S. Cl. CPC A61K 31/7048
USPC 514/27

(57) ABSTRACT

The present invention relates to methods for decreasing intestinal glucose uptake and inducing incretin release in a human subject, entailing the activation of human G protein-coupled receptor GPR40 (a receptor expressed in human intestinal L-cells) by administering one or more anthocyanin or anthocyanidin compounds, preferably one or more delphinidin compounds, in an amount effective for the activation of GPR40 and the induction of incretin.

### RELATED APPLICATIONS

This application claims priority from U.S. Ser. No. 61/721738, filed 4 Nov. 2012.

### GOVERNMENT INTEREST

None.

### BACKGROUND OF THE INVENTION

1. Field of the Invention

The present invention relates to methods for the activation of G protein-coupled receptor GPR40 expressed in intestinal L-cells. The method considers different compounds and/or compositions comprising said compounds, which act as agonists of the GPR40 receptor. The disclosure includes methods for increasing incretin secretion. The compounds or compositions comprising said compounds used in the method for the activation of GPR40, are anthocyanins, anthocyanidins, and more particularly delphinidin, or mixtures thereof. The compounds or mixtures thereof can be obtained from an extract of a plant or fruit containing said molecules. More particularly, the invention relates to extracts of berry plants and fruits, particularly Açaí, Barberry (Berberis), Bearberry (Arctostaphylos spp.), Bilberry, Blackberry, including many species and hybrids, Blueberry, Cherry, Cloudberry, Coffee berries, Cowberry (Vaccinium vitis-idaea), Cranberry, Crowberry (Empetrum spp.), Currant (Ribes spp.) red, black, and white types, Dewberry, Elderberry (Sambucus niger), Falberry, Gooseberry (Ribes spp.), Grape (Vitis vinifera), Hackberry (Celtis spp.), Honeysuckle (Lonicera spp.), Huckleberry, Indian gooseberry (Phyllanthus emblica), Lingonberry, Loganberry, Maqui (Aristotelia chilensis), Mayapple, Mulberry, including red and white mulberry, Nannyberry or sheepberry (Viburnum spp.), Oregon-grape (Mahonia aquifolium), Palqui (Cestrum parqui), Raspberry of several species, Salmonberry, Sea Grape, Sea-buckthorn, Strawberry (Fragaria), Strawberry tree (Arbutus unedo), Thimbleberry, Ugniberry, Wineberry, Wolfberry, Magelian Barberry (calafate) (berberis microphylla, berberis buxifolia, berberis heterophylla), and Chilean guava (strawberry myrthe, ugniberry, New Zealand cranberry, tazzyberry, Ugni molinae fruit extracts.

2. Description of Background

Free Fatty Acids (FFAs) regulate the release of insulin from beta-cells in the pancreas. The GPR40 receptor, formerly known as Free Fatty Acid 1 Receptor (FFA1) is a G-protein coupled receptor (GPCR). Medium to long chain free fatty acids act as agonists for this receptor. This receptor is expressed in skeletal muscle, heart, liver, and pancreatic beta-cells. Our invention entails using anthocyanins or anthocyanidins to regulate the GPR40 receptor.

As it has been known for some time, the bioavailability of anthocyanins in the blood stream is reduced (Shipp J and El-Sayed M. Abdel-Aal. (2010). Food applications and physiological effects of anthocyanins as functional food ingredients are described in The Open Food Science Journal vol., 4, 7-22. Several studies show that anthocyanins are poorly absorbed from the gastrointestinal tract, distributed to the circulatory system and excreted in the urine. These publications teach that only a small percentage, less than 0.1%, of the consumed anthocyanins is reported to be excreted in the urine. Other publications indicate that the anthocyanins enter in direct contact with the surface of stomach or intestine when ingested (Talavera S, Felgines C, Texier O, Besson C, Lamaison J-L, Remesy C. (2003). Anthocyanins are efficiently absorbed from the stomach in anesthetized rats. J Nutr. Vol., 133: 4178-4182; and Keppler K et al, 2005 “Metabolism of anthocyanins and their phenolic degradation products by the intestinal microflora” Bioorg Med Chem. Vol., 13: 5195-5205).

Thus, the compounds and compositions of the present invention are not working as an insulin secretagogue, as the molecules described in the cited documents, but the effect of the molecules and compositions of the present invention is exerting its agonist effect at the intestine level, where a decrease of intestinal glucose transport is achieved together with the activation of GPR40 of intestinal L-cells, inducing the release of incretins, such as GLP-1.

3. Objects

It is an object of the present invention to provide compounds, compositions, and methods which are useful in the activation of GPR40 present in intestinal L-cells. In particular, it is an object of the invention decreasing the intestinal glucose transport and a second particular object of the invention is increasing the release of incretins in general, more particularly GLP-1, from intestinal L-cells.

### SUMMARY

The present invention is related to compounds, compositions and methods which allow the decrease of intestinal glucose transport and the increase of incretins in general, more particularly GLP-1, from intestinal L-cells.

The compounds are selected among anthocyanins, anthocyanidins, and more particularly delphinidins. The compounds or mixtures thereof can be obtained from an extract of a plant or fruit containing said molecules. For example, the claimed compounds may be extracted from berry plants and fruits, particularly Açaí, Barberry (Berberis), Bearberry (Arctostaphylos spp.), Bilberry, Blackberry, including many species and hybrids, Blueberry, Cherry, Cloudberry, Coffee berries, Cowberry (Vaccinium vitis-idaea), Cranberry, Crowberry (Empetrum spp.), Currant (Ribes spp.) red, black, and white types, Dewberry, Elderberry (Sambucus niger), Falberry, Gooseberry (Ribes spp.), Grape (Vitis vinifera), Hackberry (Celtis spp.), Honeysuckle (Lonicera spp.), Huckleberry, Indian gooseberry (Phyllanthus emblica), Lingonberry, Loganberry, Maqui (Aristotelia chilensis), Mayapple, Mulberry, including red and white mulberry, Nannyberry or sheepberry (Viburnum spp.), Oregon-grape (Mahonia aquifolium), Palqui (Cestrum parqui), Raspberry of several species, Salmonberry, Sea Grape, Sea-buckthorn, Strawberry (Fragaria), Strawberry tree (Arbutus unedo), Thimbleberry, Ugniberry, Wineberry, Wolfberry fruit extracts, Magelian Barberry (calafate) (berberis microphylla, berberis buxifolia, berberis heterophylla), and Chilean guava (strawberry myrthe, ugniberry, New Zealand cranberry, tazzyberry, Ugni molinae fruit extracts. The claimed compositions may comprise molecules extracted from one or more of the mentioned plant species, together with pharmaceutical adjuvants, carriers, and/or excipients.

The compounds and/or compositions of the present invention allow the maintenance of glucose levels in a patient by controlling two different aspects. The first aspect, a short-term acting mechanism, corresponds to a decrease in intestinal glucose transport, thus, avoiding the entry of ingested glucose to the blood stream. In this regard, it is known that in the intestine, the enterocytes of the brush border membrane (BBM) are the primary site of absorption of dietary sugars. The co-transporter Na⁺/glucose SGLT-1 transports glucose and galactose from the lumen of the intestine into the enterocytes (Ducroc R et al “Luminal leptin induces rapid inhibition of active intestinal absorption of glucose mediated by sodium-glucose cotransporter 1”, Diabetes 2005; 54:348-354). SGLT-1 is a functional part of peripheral system that senses exogenous glucose to maintain energy homeostasis (Inigo C et al “Luminal leptin inhibits intestinal sugar absorption in vivo”, Acta Physiol 2007; 190:303-310). The SGLT-1 activity is highly regulated by several peptides and hormones active in the membranes of the mucosa or serosa (Hardin J A et al “Effect of luminal epidermal growth factor on enterocyte glucose and proline transport” Am J Physiol 1996; 271:G509-G515; Wong T P et al “Involvement of an enterocyte renin angiotensin system in the local control of SGLT1-dependent glucose uptake across the rat small intestinal brush border membrane” J Physiol 2007; 584:613-623; and Yamauchi et al, “Altered expression of goblet cell- and mucin glycosylation-related genes in the intestinal epithelium during infection with the nematode Nippostrongylus brasiliensis in rat” APMIS 2008; 114:270-278). The regulation of SGLT-1 involves a postprandial mechanism, followed by a rapid adjustment of the abundance of the protein in the BBM and recruitment of glucose transporter GLUT-2, which depends on the levels of luminal sugars (Inigo et al, 2007). The activity of SGLT-1 and GLUT-2 is increased in type II diabetes (Kellett G L et al “The diffusive component of intestinal glucose absorption is mediated by the glucose-induced recruitment of GLUT2 to the brush-border membrane” Biochem J 2000; 350:155-162; and Kushiyama A et al, “Resistin-like molecule activates MAPKs, suppresses insulin signaling in hepatocytes, and induces diabetes, hyperlipidemia, and fatty liver in transgenic mice on a high fat diet” J Biol Chem 2005; 280:42016-42025) probably as a result of disregulation. The compounds and compositions of the present invention, as it will be shown in the Examples section, inhibit the glucose transport (SGLT-1) in jejunal mucosa, thus, demonstrating the effectiveness of the compounds and compositions of the present invention in decreasing the intestinal glucose transport, i.e., the first mentioned aspect.

A second aspect, which is a medium-term acting mechanism, corresponds to the activation of GPR40 receptors of intestinal L-cells. More particularly, the activation of the GPR40 of L-cells stimulates the secretion of incretins, more specifically GLP-1, which is released into the circulatory system. The release of GLP-1 into the bloodstream permits a wider action of the molecules and/or compositions of the present invention, allowing for example, the control of gastric emptying, decrease of glucose production (gluconeogenesis) in the liver, higher glucose uptake in the muscles, increase of insulin secretion and decrease of glucagon secretion in the pancreas, among others, which finally produces an integral control of glucose concentration in blood. All of this cannot be achieved by GPR40 agonists which are absorbed and incorporated directly into blood stream, since the GPR40 agonist effect is focalized in GPR40 present in beta-cells of the pancreas, thus, having a much reduced effect, and not an integral effect as the one achieved with the compounds and compositions of the present invention. On the other hand, it is known that GPR40 is expressed in enteoendocrine cells and mediates free fatty acid stimulation of incretin secretion (Edfalk et al, Diabetes 2280-7, 2008). Our invention proposes that our composition and methods activates incretins via GPR40 receptors located in L cells.

### BRIEF DESCRIPTION OF THE FIGURES

FIG. 1: Measurement of plasmid-expressed 5′ adenosine monophosphate-activated protein kinase isoform 1β1 (pAMPK1β1) and β-actin (as control) over time.

FIG. 2: Effect of Delphinidin on the transport of D-glucose coupled to sodium in mouse jejunum. A) Voltage recording transepithelial (Vₜₑ) of a piece of mouse jejunum mounted in Using Chamber. 1 indicates the time when D-glucose in the mucosal side is increased from 0 to 10 mM. Vₜₑ negative deflection reflects the electrogenic transport of D-glucose coupled to sodium, where the cations accompanying the monosaccharide reach the serosal side to be actively transported by the Na⁺/K⁺ ATPase. At 2 shows the time when 50 μM delphinidin is added to the mucosal side, causing a positive deflection in voltage that can be caused by a decrease in glucose transport coupled to sodium. (B) Summary of Δ current for the exemplified experiments. (A). Delphinidin decreases about 40% (ii: delphinidin sensitive current) of the sodium current induced by 10 mM D-glucose (i). Values are mean±error standard, n=4 different animals.

FIG. 3: Delphinidin and Cyanidin induces calcium fluxes in HT-29 cells trough GPR40 receptor. Fura-2/AM-loaded HT-29 cells were suspended in Ca⁻²-HEPES buffer and then stimulated with delphinidin or cyanidin. Representative measurements of the effects induced by 100 μM delphinidin or 100 μM Cyanidin on the 340/380 ratio. The contribution of GPR40 receptor on the increase of calcium influx induced by delphinidin or cyanidin, was evaluated using GW1110 which is an antagonist of this receptor.

FIG. 4: GW110 blocks GLP-1 mRNA expression induced by a composition of the invention NCI-HT16 cells. The effect of treatment with GW1100 (10 μM) GLP-1 mRNA expression induced by a composition of the invention was studied. NCI-H716 cells were incubated with 10 μM GW1100 and then stimulated with a composition of the invention (0.5, 5, 50, 500 μg/mL) for 24 hr. RNA was extracted and subjected to analysis by qRT-PCR.

FIG. 5: Delphinidin induces GLP-1 mRNA expression. NCI-H716 cells were incubated with 10, 50 or 100 μM delphinidin for 24 hr and then RNA was extracted and subjected to analysis by qRT-PCR.

FIG. 6: GW110 block delphinidin-induced GLP-1 production. NCI-H716 cells were pre-incubated with 10 μM GW1100 and then stimulated with 50 μM delphinidin for 48 hr in presence of Brefeldin A. The amount of GLP-1 was measurement trough flow cytometry.

FIG. 7: A composition of the invention decreased basal hyperglycemia concentration in diabetic rats. STZ induced diabetic rats were treated with 20 mg/Kg of a composition of the invention for 4 months and glucose concentration was determined in serum by enzymatic method of glucose oxidase. The results are the means ±error standard, n=5. *P<0.001.

FIG. 8: Effect of a composition of the invention on postprandial blood glucose levels in normal control and diabetic rats induced by STZ injection. Glucose tolerance test. (A) Blood glucose levels in normal control rats (NC) and normal control rats treated with vehicle or 20 mg/Kg of a composition of the invention, respectively. (B) Blood glucose levels in the diabetic rats (DR) and diabetic rats treated with vehicle or 20 mg/Kg of a composition of the invention, respectively. The results are the means±error standard, n=5. *Different from DR, P<0.05. D and G indicate the administration of a composition of the invention and glucose, respectively.

FIG. 9: Effect of a composition of the invention on postprandial insulin and glucose concentrations in human test subjects with mild glucose intolerance.

### DESCRIPTION OF THE PREFERRED EMBODIMENTS

The present invention considers compounds and compositions for the decrease of intestinal glucose transport and activation of intestinal L-cells for release of incretins in general, more particularly GLP-1.

In a particular embodiment, the compounds are selected from among anthocyanidin derivatives (“anthocyanidins”) such as anthocyanins. The general structure of these classes of compounds is as shown:

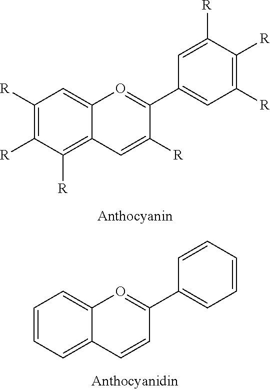

More particularly, I prefer delphinidin (i.e., 2-(3,4,5-trihydroxyphenyl)chromenylium-3,5,7-triol) and derivatives thereof (e.g., delphinidin glycosides).

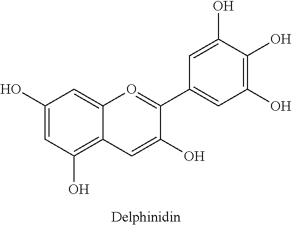

In a further embodiment, the invention comprises compositions containing one or more molecules selected from anthocyanidin derivatives (“anthocyanidins”) e.g., anthocyanins, and preferably delphinidin and derivatives thereof (“delphinidins”), or mixtures thereof.

In a more specific embodiment, the compositions of the present invention comprise a combination of eight selected anthocyanins in particular amounts. The anthocyanindins comprised in the compositions of the preferred embodiment are delphinidin-3-O-sambubioside-5-O-glucoside, delphinidin-3,5-O-diglucoside, cyanidin-3-O-sambubioside-5-O-glucoside, cyanidin-3,5-O-diglucoside, delphinidin-3-O-sambubioside, delphinidin-3-O-glucoside, cyanidin-3-O-sambubioside and cyanidin-3-O-glucoside.

Preferably, the invention features compositions that comprise at least about 35% of the aforementioned eight anthocyanins(delphinidin-3-O-sambubioside-5-O-glucoside, delphinidin-3,5-O-diglucoside, cyanidin-3-O-sambubioside-5-O-glucoside, cyanidin-3,5-O-diglucoside, delphinidin-3-O-sambubioside, delphinidin-3-O-glucoside, cyanidin-3-O-sambubioside, and cyanidin-3-O-glucoside) by weight in their constitution, wherein at least about 28% of the weight of the compositions corresponds to delphinidins. These compositions are not naturally occurring, and are non-toxic.

In a preferred embodiment, the compositions of the present invention comprise the following amount in weight of each anthocyanin: at least about 6% by weight of delphinidin-3-O-sambubioside-5-O-glucoside, at least or about 13% by weight of delphinidin-3,5-O-diglucoside, at least about 3% by weight of cyanidin-3-O-sambubioside-5-O-glucoside, at least or about 1% by weight of cyanidin-3,5-O-diglucoside, at least about 1% by weight of delphinidin-3-O-sambubioside, at least about 6% by weight of delphinidin-3-O-glucoside, at least about 0,7% by weight of cyanidin-3-O-sambubioside, at least about 1% by weight of cyanidin-3-O-glucoside).

The non-naturally occurring, standardized and rationally designed compositions, comprising four different delphinidins and four different cyanidins, of the present invention are different from others in prior art, due to their particular selection of delphinidins and cyanidins, their high concentration of anthocyanins, particularly delphinidins, and their method of preparation, which guarantee to contain each compound in the same amount in each batch, therefore, also the effects are always the same.

The compounds or mixtures considered in the present invention can be obtained from an extract of a plant or fruit containing said molecules. More particularly, the invention relates to extracts of berry plants and fruits, particularly Açaí, Barberry (Berberis), Bearberry (Arctostaphylos spp.), Bilberry, Blackberry, including many species and hybrids, Blueberry, Cherry, Cloudberry, Coffee berries, Cowberry (Vaccinium vitis-idaea), Cranberry, Crowberry (Empetrum spp.), Currant (Ribes spp.) red, black, and white types, Dewberry, Elderberry (Sambucus niger), Falberry, Gooseberry (Ribes spp.), Grape (Vitis vinifera), Hackberry (Celtis spp.), Honeysuckle (Lonicera spp.), Huckleberry, Indian gooseberry (Phyllanthus emblica), Lingonberry, Loganberry, Maqui (Aristotelia chilensis), Mayapple, Mulberry, including red and white mulberry, Nannyberry or sheepberry (Viburnum spp.), Oregon-grape (Mahonia aquifolium), Palqui (Cestrum parqui), Raspberry of several species, Salmonberry, Sea Grape, Sea-buckthorn, Strawberry (Fragaria), Strawberry tree (Arbutus unedo), Thimbleberry, Ugniberry, Wineberry, Wolfberry, Magellan Barberry (calafate) (berberis microphylla, berberis buxifolia, berberis heterophylla), and Chilean guava (strawberry myrthe, ugniberry, New Zealand cranberry, tazzyberry, Ugni molinae fruit extracts.

### EXAMPLES

The following examples are provided in order to illustrate the invention, but in no way they are to be understood as limiting the scope of the invention. In the present disclosure, we describe Examples considering the use of a particular embodiment of the invention. In all of the following Examples, when reference is made to a composition according to the invention it must be understood as an standardized extract of Maqui berry (Aristotelia chilensis), containing not less than 25% total delphinidins.

### Example 1

### Standardized Extract of Aristotelia Chilensis Fruits

The extract was standardized to a minimum of 35% total anthocyanins and NLT 25% of total delphinidins. Table 1 summarizes the content of a composition according to the invention, wherein the total of the components sum up to 35.2%, demonstrating the standardization of the composition. When reference is made to delphinidin in the Examples, delphinidin (>98% pure) was purchased from Extrasynthase S. A.(Lyon, France).

TABLE 1
Content of a composition according to the invention.

| Component | Content [%] |
|---|---|
| Delphinidin-3-O-samb-5-O-gluc | 6.38 |
| Delphinidin-3,5-O-diglucos | 13.64 |
| Cyanidin-3-O-samb-5-O-gluc | 3.36 |
| Cyanidin-3,5-O-diglucos | 1.58 |
| Delphinidin-3-O-sambubioside | 1.67 |
| Delphinidin-3-O-glucoside | 6.95 |
| Cyanidin-3-O-sambubioside | 0.79 |
| Cyanidin-3-O-glucoside | 1.05 |
| Total: | 35.42 |

### Example 2

### Electric Absorption Studies of D-Glucose in the Small Intestine

C57B1/6J mice were obtained from The Jackson Laboratory (Bar Harbor, Me., USA). The mice were maintained at the Specific Pathogen Free mouse facility of the Centro de Estudios Científicos (CECS), Valdivia, Chile and prior to the experiments they had free access to water and food. Animals were killed by cervical dislocation according to local Institutional Animal Care and Use Committee (IACUC) regulations. The jejunum was isolated, open longitudinally along the mesenteric border and rinsed with phosphate buffered saline (PBS).

Two sections of jejunum per animal were mounted in Ussing Chambers and maintained in Ussing buffer (120 Mm NaCl, 25 mM NaHCO₃, KH₂PO₄ 3.3 mM, 0.8 mM K₂HPO₄, 1.2 mM MgCl₂ and 1.2 mM CaCl₂) supplemented with 10 mM D-glucose in the serosal side. The temperature was maintained at 37° C. and the solution was continuously gassed with carbogen 5% CO₂. Once the preparation reached a stable record of electrical parameters, 10 mM D-glucose was added to the mucosal side of the preparation in order to stimulate the transport of D-glucose coupled to Na⁺. The effect of delphinidin on sodium-coupled transport of glucose was determined by application of this molecule at a concentration of 50 μM in the mucosal side of the preparation. The transepithelial electrical potential difference (VM), defined as the potential side compared to serous mucus preparation was recorded continuously under current clamp configuration using a VCC MC2 amplifier (Physiological Instruments). The values of short circuit current (I_{sc}) were calculated from experimental data using Ohm's law. The results are expressed as the intensity of the I_{sc} (μA/cm²).

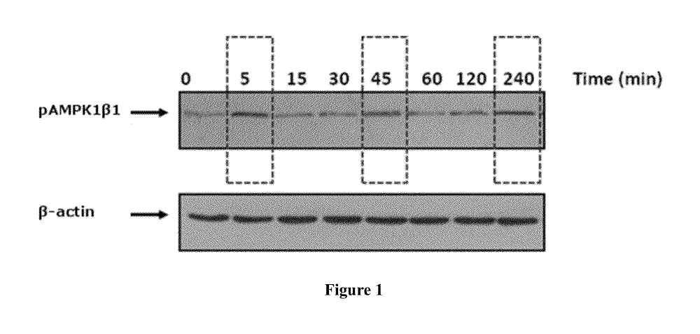

It i is known that in intestine the glucose entry can involve active Na⁺-dependent GLUT (SGLT-1) or diffusive transporter GLUT2. We used the Ussing chamber measurements to characterize the effect of mucosal delphinidin on rat intestinal active glucose transport (SGLT-1). Mice jejunal mucosa mounted in the chamber was allowed to reach a steady state. The addition of 10 mM glucose in the mucosal bath induced a rise in I_{sc} (maximum after 3 min) representing an increase in SGLT-1 activity. When pure delphinidin was added to the mucosal bath it induced a marked inhibition of glucose-induced I_{sc} (FIG. 1A) inhibiting glucose transport by jejunal mucosa in approximately 60% (FIG. 1B).

### Example 3

### Measurement of Calcium Influx

HT-29 cells were suspended in HBSS at a concentration of 20×10⁶ cells/ml and incubated with 1 μM Fura-2/AM (Molecular Probes) for 30 min at 37° C. The cells were then washed twice, divided into 2×10⁶ cells/ml aliquots and maintained at 4° C. in the dark until use. Cells were then pelleted by centrifugation at 1200 rpm for 6 min and re-suspended in 2 ml HEPES buffer [20 mM HEPES, pH 7.2; 140 mM NaCl; 10 mM glucose; 1 mM KCl; 1 mM Ca²⁺ chloride (CaCl₂); and 1 mM MgCl₂].

To assess and ensure the participation of GPR40 on calcium mobilization induced by delphinidin and cyanidin, Fura-2/AM-loaded HT-29 cells (2×10⁶ cells/ml) were suspended in Ca²⁺-HEPES buffer. At 80 s, delphinidin or cyanidin (100 μM) were added. The influx of Ca²⁺ was recorded as the 340:380 excitation ratio at 509 nm emission in a LS55 thermoregulated spectrofluorimeter (Perkin-Elmer, Mass., USA).

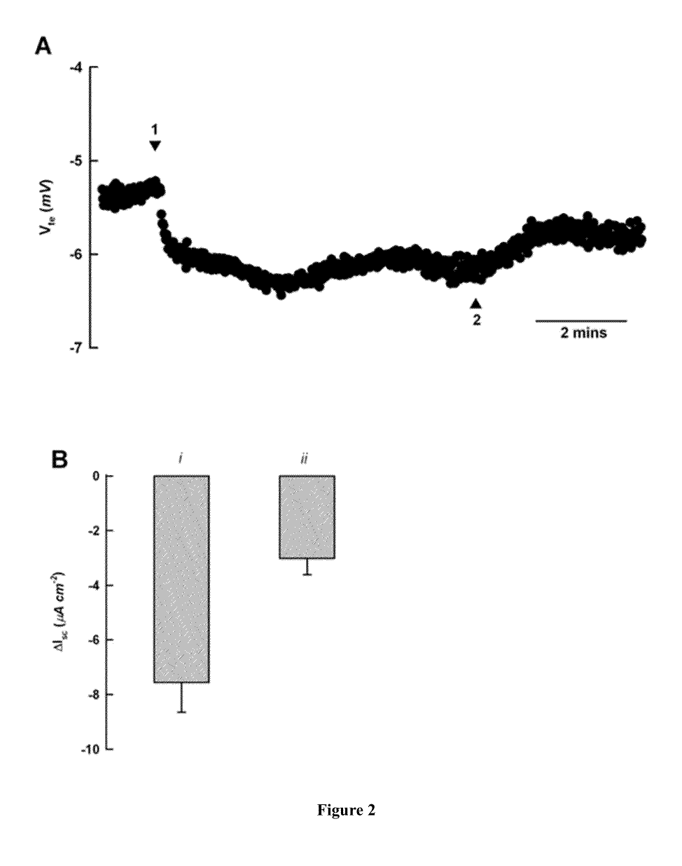

To evaluate the contribution of GPR40 receptor on the increase of calcium influx induced by delphinidin or cyanidin, GW1110, which is an antagonist of this receptor, was used. In GW1100 significantly reduced cyaniding- and delphinidin-induced calcium flux in Fura-2/AM-loaded HT-29 cells (FIGS. 2A and 2B).

### Example 4

### Incretin Release Secretion by a Composition of the Invention and Delphinidin

Total RNA was isolated from NCI-HT16 cells using the RNeasy Plus Mini Kit according to the manufacturer's instructions. Approximately 1 μg of total RNA, 0.5 μg of oligo (dT)₁₈ primer, 10 mM dNTPs and 200 U of reverse transcriptase were used for each cDNA synthesis reaction. PCR amplifications were performed using the Brilliant II SYBR Green QPCR Master Mix (Stratagene) according to the manufacturer's instructions. All reactions were carried out using an Mx300p thermal cycler (Stratagene, Calif., USA). GLP-1 specific primers were used.

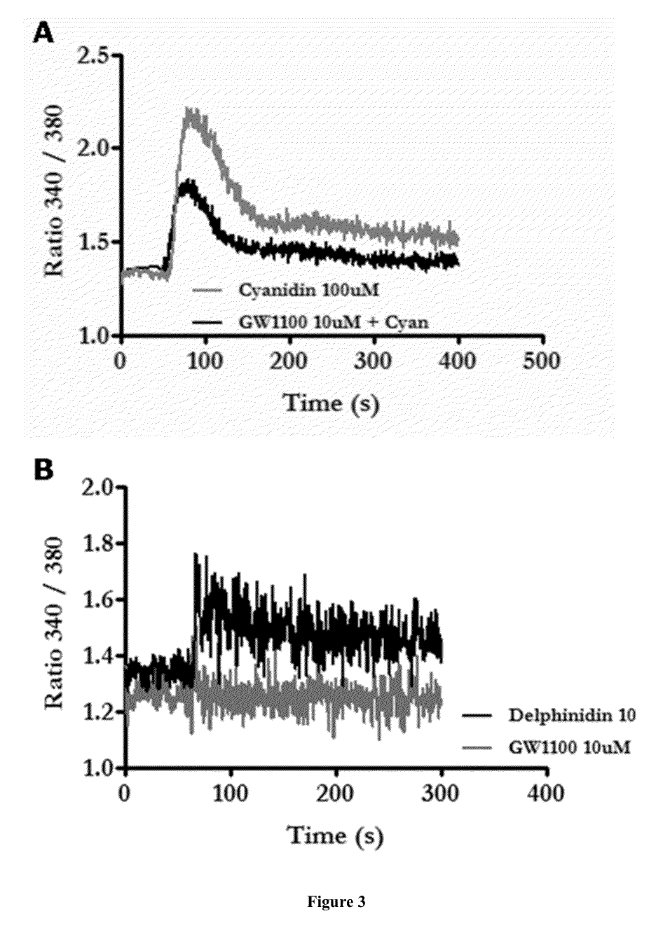

A composition of the invention increased the expression of GLP-1 mRNA after 24 h of incubation. To assess if GPR40 receptor is involved in GLP-1 production induced by a composition of the invention, NCI-HT16 cells were preincubated with GW1100 prior to stimulation with a composition of the invention. GW1100 pretreatment inhibited GLP-1 mRNA expression, indicating that GPR40 receptor mediated the GLP-1 mRNA expression in this cellular type (FIG. 3).

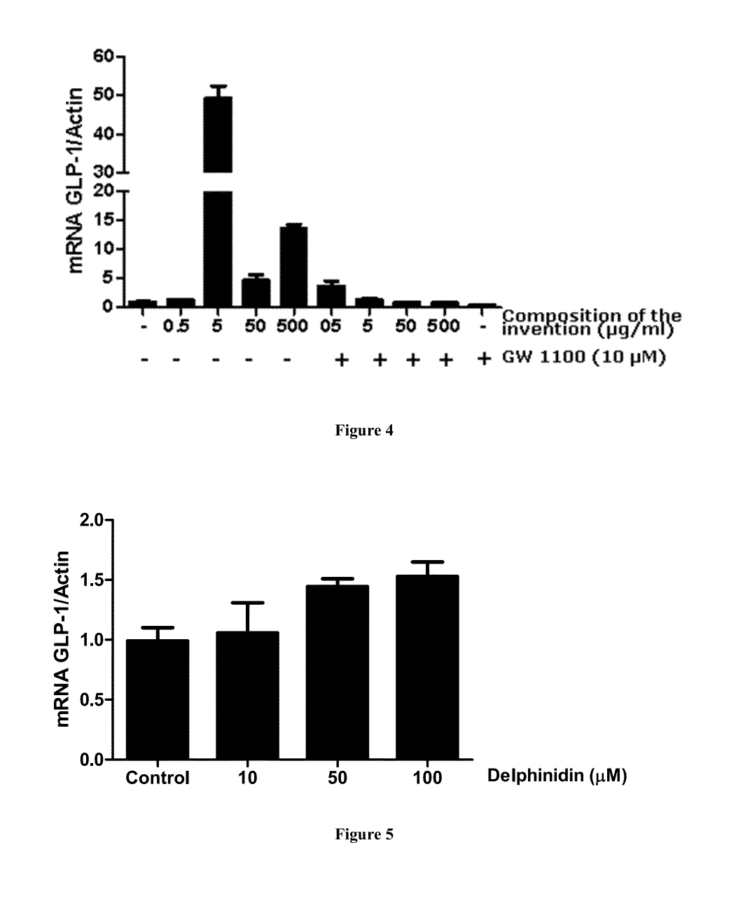

Delphinidin increased the expression of GLP-1 mRNA after 24 h of incubation at concentration of the 50 and 100 μM with respect to control (FIG. 4).

For the detection of GLP-1 production by flow cytometry the NCI-H716 cells (1×10⁶ cells/mL) were pre-incubated with 10 μM GW1100 for 30 minutes and then stimulated with 50 μM delphinidin for 48 hrs in presence of Brefeldin A. After the incubation period, the cells were washed twice with PBS. After two washes in PBS, the cells were stained with mouse anti-GLP-1 antibody for 1 hr at 4° C. The cells were washed twice with PBS, incubated for 1 hr at 4° C. with anti-mouse Alexa 488 (1:200 dilution) and finally washed twice with PBS. The cells were assessed by flow cytometry on a FACSCanto II (Becton Dickinson).

To assess if GPR40 receptor is involved in delphinidin-induced GLP-1 production, we analyzed the effect of GW1100 on the GLP-1 production induced by 50 μM Delphinidin. First, the flow cytometric analysis of GLP-1 production in NCI-H716 cells showed that delphinidin induced an increase in the amount of GLP-1 compared to control cells and that GPR40 receptor mediated GLP-1 production induced by delphinidin, because GW100 decreases the production of this incretin (FIG. 5).

### Example 5

### Effect of a Composition of the Invention in Glucose Tolerance Test (GTT) in Rats

Diabetes was induced by a single tail injection of STZ (Calbiochem, Darmstadt, Germany) dissolved in citrate buffer (pH 4.5) at a dose of 55 mg/Kg in male Rattus norvegicus (250-300 grs). Normal control animals (NC, n=5) were fasted overnight and injected with the citrate buffer vehicle.

Diabetic animals were divided into two groups. Group I (n=5) was studied after sixteen weeks of untreated diabetes. Group II was studied after sixteen weeks of untreated diabetes and then administered 20 mg/Kg (n=5) of a composition of the invention. All animals were fed standard rat laboratory diet and water ad libitum. Animals were provided by the Pontificia Universidad Católica of Chile and maintained on a 12-h light-dark cycle at 22° C.

Oral glucose tolerance test (GTT) was performed 30 minutes after the treatment with a composition of the invention in normal control and diabetic rats induced by STZ injection. Glucose (2.0 g·kg⁻¹ body weight) was administered via intraperitoneal injection after 12-h fasting. Blood se was determined at 0, 30, 60, 120 and 240 minutes after glucose challenge using the enzymatic method of glucose oxidase (Wiener lab) at 490 nm.

To establish possible differences between the plasmatic parameters for each product in each volunteer, the multifactorial variance test (ANOVA) was used with a statistical significance of p=0.05. The variation parameters considered in the evaluation were: administered product, period of administration, sequence and residual effect. The protocol followed the recommendations and Guidelines of the FDA, both for the design and pharmacokinetic and statistical analysis of the study.

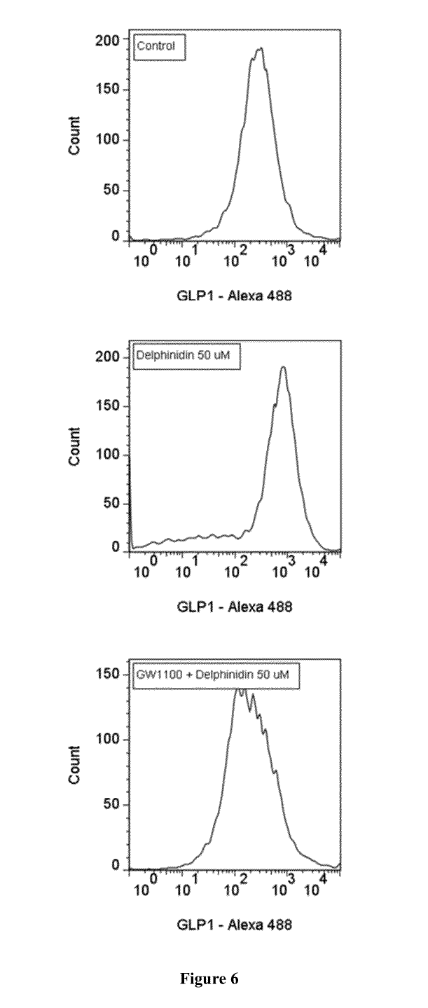

A composition of the invention decreased significantly basal serum glucose concentration in STZ induced diabetic rats with respect to the diabetic control group, after 4 months of treatment at a concentration of 20 mg/Kg (FIG. 6). The blood glucose concentration decreased between the diabetic control group (100.25±11.6 mg/dL) and diabetic rats group (475±91.3 mg/dL) treated with a composition of the invention in approximately 4 fold. Moreover, the control rats groups treated with the composition of the invention were unchanged in blood glucose concentration (FIG. 6).

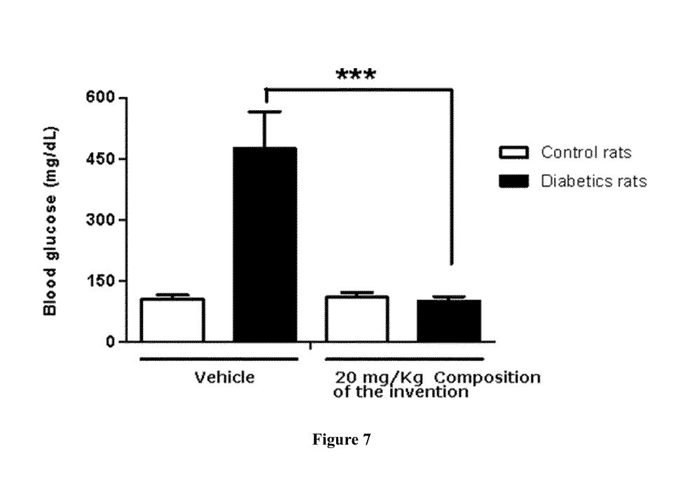

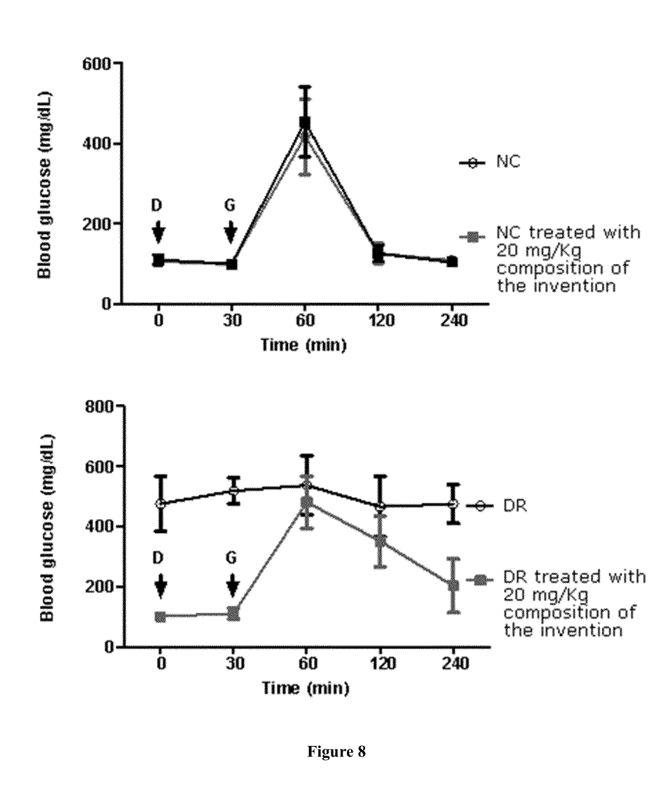

The composition of the invention was also evaluated in the glucose tolerance test and administered 30 minutes (2.0 g·kg⁻¹ body weight) before time evaluation. In normal control rats treated with 20 mg/Kg of the composition of the invention, we did not observed changes in the blood glucose levels after the administration of glucose via intraperitoneal injection (FIG. 7A). However, in diabetic rats treated with 20 mg/Kg of the composition of the invention, we observed a decreased of the basal glucose levels (FIG. 7B). The results of the glucose tolerance test clearly showed that the composition of the invention ameliorates insulin resistance in the diabetic group. The glucose-lowering effect was significantly greater in the diabetic rats group treated with the composition of the invention at 240 min after glucose administration (FIG. 7).

Recently, considerable attention has focused on dietary constituents that may be beneficial for the prevention and treatment of diabetes. Although there are some drugs that have been used as therapeutic regimens for obesity related metabolic diseases, there is little evidence that food factors themselves can be directly beneficial for modulating insulin sensitivity.

Triglycerides levels in diabetic rats treated with the composition of the invention remained unchanged with respect to the diabetic control group (data not shown).

The results demonstrate that the composition of the invention reduces basal hyperglycemia and enhances the ability to metabolize glucose in type 2 diabetic rats.

### Example 6

### Effect of the Composition of the Invention in Human Patients

The objective of this human clinical study was to evaluate the efficacy of a single oral administration of the composition of the invention on the postprandial levels of glucose and insulin and tolerability in individuals with glucose intolerance.

We used a randomized, double-blind, placebo-controlled crossover study design that was approved by the Scientific Ethical Committee of the Occidental Metropolitan Health Service, Ministry of Health, Chile, including insurance policy for the patients at Hospital Clínico de la Universidad de Chile, Prof. José Joaquin Aguirre; Hospital San Juan de Dios. The study was conducted at Centro de Investigaciones Farmacológicas y Toxicológicas (IFT), Facultad de Medicina. Universidad de Chile.

Twelve volunteers patient adults of both sexes were enrolled and were divided in 2 groups of 6. Placebo and treatment phases were separated by a 7-day washout period. Subjects on recruitment were ages between 18 and 55 years, not receiving any acute or chronic pharmacological therapy, had a body mass index (BMI) inferior to 30 kg/m², a fasting plasmatic glucose (BG) <110 mg/100 mL and an altered glucose tolerance test (110 to 125 mg/1100 ml), defined as plasma glucose between 100 mg/100 mL<125 mg/100 ml (o<5.6-<7.8 mmol/L), 120 minutes after the intake of 75 grams of carbohydrates (cooked white rice, grade 1).

Patients with antecedents of drug and/or alcohol abuse and smokers (more than 3 cigarettes every 7 days) were excluded from the study. Also excluded were subjects taking vitamin supplements 7 days prior to the administration of the test products, subjects with a recent change in the food habits or exercise, and subjects using chronic pharmacological therapy (except single dose of over the counter medicines) or medicines that affect hepatic enzymatic activity 28 days prior to the beginning of the study, subjects with allergies to any medicine or with diabetes mellitus or hospitalized during the last 60 days, or with arrhythmia and renal insufficiency (blood creatinine>1.5 mg/dl), or known allergy to peanuts and almonds., or any other chronic or limiting disease including alcoholism or any disease or condition that the physician considers that the volunteer does not qualify for participating in the trial.

All appointments for the 12 patients were arranged at PTRC. All patients fulfilled the inclusion criteria. The included patients were randomized into active or placebo groups based on the order of their chosen date and the arrival time for their post-screening clinic.

The appearances of the test product and placebo were identical, and no aroma or visual recognition was possible from either. Achievement of blindness was validated before the trial in a group of 10 volunteers. At the completion of the study, we conducted a simple survey asking the subjects to guess whether they were in the study product or placebo group in order to evaluate the extent of study blindness.

In each session, each test subject fasted for 12 hours. Each then received a glass of water (250 ml) containing either a placebo or 200 g of the composition described in Example 1 dissolved in water as a single dose. Thirty minutes after administration of the test composition or placebo, each test subject was fed 75 g cooked white rice grade I, prepared by a nutritionist of the study.

Several blood samples were obtained in each session from each test subject. The first blood sample was taken as the base line 10 minutes before (time −10) the treatment with the composition of the invention or placebo (time 0). A second blood sample was drawn 15 minutes after intake of the product or placebo (time +15). The rice was served 30 minutes after the intake of the composition of the invention or placebo, the third sample obtained at the same moment (time +30). Then, consecutive blood samples were taken at times, +60, +90, and +120 and +180 minutes after the intake of the product or placebo.

These blood samples were used to calculate using the blind individual crossover (“Standard Glucose Tolerance Test”). Glucose was measured in plasma by the photo-enzymatic method GOD-PAP (Diasys) and insulin measured in blood (plasma) in all samples by an immune assay method MEIA (Abbott).

Adverse reactions were assessed by a physician that recorded observations during the treatment, and classified them as few, moderate or severe and filled a form with the general state of health of the patient.

The process was repeated four times with each test subject. The test subjects were then crossed over, subjects who had received placebo now were administered test compound, and subjects who had received test compound now received placebo. The wash out period between the crossover administrations was 6 days.

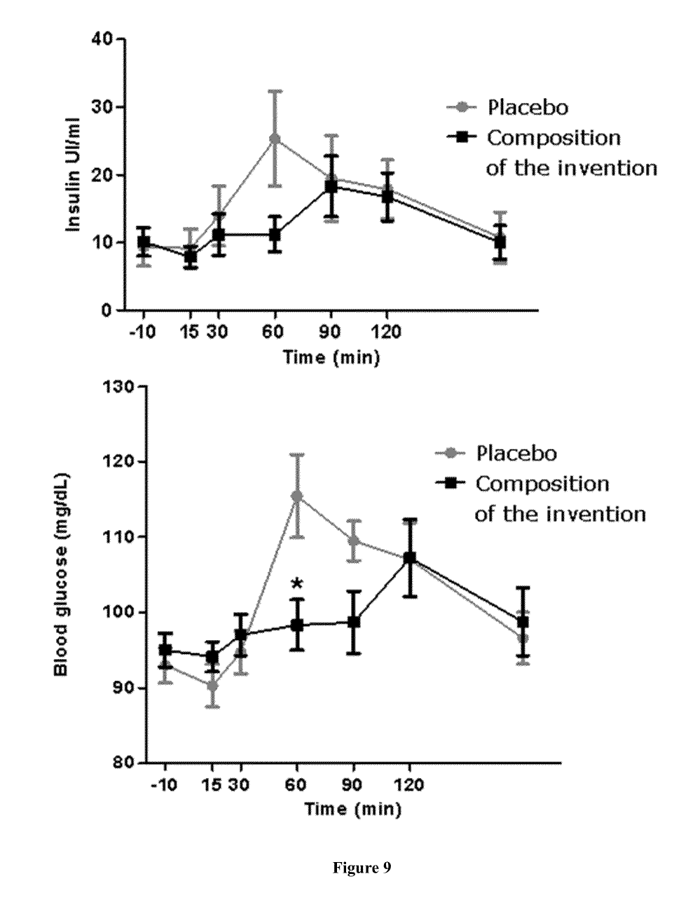

The results show that a single administration of the composition of the invention depresses postprandial levels of insulin (FIG. 9A) and glucose (FIG. 9B) viz levels produced by placebo.

FIG. 9 shows the effect of the composition of Example 1 on postprandial insulin and glucose concentrations in patients with mild glucose intolerance, using a glucose tolerance test. Each time, both groups (placebo and a composition of the invention), received 30 minutes before a meal (defined and a fixed quantity of 75 g cooked white rice (Grade 1). Pre-prandial plasmatic glucose was measured at basal time (time −10 minutes) and the post-prandial concentrations (at the end of time 180 minutes) were calculated using the blind individual crossover (“Standard Glucose Tolerance Test). Blood samples were obtained in each session. The results are the means±error standard, n=8. *Different from Placebo, P<0.05.

These data show that patients who received 200 mg, of the composition of the invention show significantly reduced blood glucose levels after 30 minutes following administration of a meal, compared to patients receiving only placebo (FIG. 9B). The effect on glucose balance of the composition of Example 1 is not, however, associated with an increase in insulin secretion. See FIG. 9A. Without intending to be hound by any theoretical mechanism, I believe my composition works via an integral mechanism which considers a decrease in intestinal glucose uptake and the activation of GPR40 in intestine L-cells inducing the release of GLP-1.

Also, it was possible to observe an interesting event and kinetic results showing a shift in the time glucose and insulin reached the maximum in patients with the extract (FIG. 9A), that correlated with a late decrease in the levels of glucose (FIG. 9B).

We claim:

1. A method comprising:
Diagnosing in a human patient a condition selected from: impaired glucose tolerance, impaired fasting glucose and diabetes;
Administering to said patient at least one anthocyanidin,
said administration occurring from about 30 minutes before to about contemporaneously when said patient consumes other food,
said at least one anthocyanidin administered in an amount effective to produce in a human test subject, using the fasting-fed test method described in Example 6, a lower blood insulin level or blood glucose level than is produced by placebo, when measured 60 minutes after feeding.

2. The method of claim 1, wherein said at least one anthocyanidin comprises at least one compound selected from the group consisting of: delphinidin-3-O-sambubioside-5-O-glucoside, delphinidin-3,5-O-diglucoside, cyanidin-3-O-sambubioside-5-O-glucoside, cyanidin-3,5-O-diglucoside, delphinidin-3-O-sambubioside, delphinidin-3-O-glucoside, cyanidin-3-O-sambubioside and cyanidin-3-O-glucoside.

3. The method of claim 2, wherein said at least one anthocyanidin comprises: from about 0.1% to about 25% (w/w) of delphinidin-3-O-sambubioside-5-O-glucoside, from about 1 to about 50% (w/w) of delphinidin-3,5-O-diglucoside, from about 0.1% to about 20% (w/w) of cyanidin-3-O-sambubioside-5-O-glucoside, from about 0.05% to about 20% (w/w) of cyanidin-3,5-O-diglucoside, from about 0.015% to about 15% (w/w) of delphinidin-3O-sambubioside, from about 0.1% to about 25% (w/w) of delphinidin-3-0-glucoside, from about 0.02% to about 15% (w/w) of cyanidin-3-O-sambubioside, or from about 0.05% to about 15% (w/w) of cyanidin-3-O-glucoside.

4. The method of claim 3, wherein said at least one anthocyanidin comprises: from about 0.1% to about 25% (w/w) of delphinidin-3-O-sambubioside-5-O-glucoside, from about 1 to about 50% (w/w) of delphinidin-3,5-O-diglucoside, from about 0.1% to about 20% (w/w) of cyanidin-3-O-sambubioside-5-O-glucoside, from about 0.05% to about 20% (w/w) of cyanidin-3,5-O-diglucoside, from about 0.015% to about 15% (w/w) of delphinidin-3-O-sambubioside, from about 0.1% to about 25% (w/w) of delphinidin-3-O-glucoside, from about 0.02% to about 15% (w/w) of cyanidin-3-O-sambubioside, and from about 0.05% to about 15% (w/w) of cyanidin-3-O-glucoside.

5. The method of claim 1, said anthocyanidin(s) administered in an amount of about 200 mg.

6. The method of claim 2, said anthocyanidin(s) administered in an amount of about 200 mg.

7. A method of treating a condition selected from: impaired glucose tolerance, impaired fasting glucose and diabetes, said method comprising:
Diagnosing in a human patient a condition selected from: impaired glucose tolerance, impaired fasting glucose and diabetes;
Administering to said patient at least about 200 mg of anthocyanidin(s), Said administration occurring from about 30 minutes before to about contemporaneously when said patient consumes other food.

8. The method of claim 7, wherein said anthocyanidin(s) comprises at least one compound selected from the group consisting of: delphinidin-3-O-sambubioside-5-O-glucoside, delphinidin-3,5-O-diglucoside, cyanidin-3-O-sambubioside-5-O-glucoside, cyanidin-3,5-O-diglucoside, delphinidin-3-O-sambubioside, delphinidin-3-O-glucoside, cyanidin-3-O-sambubioside and cyanidin-3-O-glucoside.

9. The method of claim 8, wherein said anthocyanidin(s) comprises: from about 0.1% to about 25% (w/w) of delphinidin-3-O-sambubioside-5-O-glucoside, from about 1 to about 50% (w/w) of delphinidin-3,5-O-diglucoside, from about 0.1% to about 20% (w/w) of cyanidin-3-O-sambubioside-5-O-glucoside, from about 0.05% to about 20% (w/w) of cyanidin-3,5-O-diglucoside, from about 0.015% to about 15% (w/w) of delphinidin-3-O-sambubioside, from about 0.1% to about 25% (w/w) of delphinidin-3-O-glucoside, from about 0.02% to about 15% (w/w) of cyanidin-3-O-sambubioside, and from about 0.05% to about 15% (w/w) of cyanidin-3-O-glucoside.

10. A method comprising:
Administering to a human at least one anthocyanidin,
said administration occurring about 30 minutes before said human consumes other food, said at least one anthocyanidin administered in an amount effective to produce in a human test subject, using the fasting-fed test method described in Example 6, a lower blood insulin level or blood glucose level than is produced by placebo, when measured 60 minutes after feeding.

11. The method of claim 10, wherein said at least one anthocyanidin comprises at least one compound selected from the group consisting of: delphinidin-3-O-sambubioside-5-O-glucoside, delphinidin-3,5-O-diglucoside, cyanidin-3-O-sambubioside-5-O-glucoside, cyanidin-3,5-O-diglucoside, delphinidin-3-O-sambubioside, delphinidin-3-O-glucoside, cyanidin-3-O-sambubioside and cyanidin-3-O-glucoside.

12. The method of claim 11, wherein said at least one anthocyanidin comprises: from about 0.1% to about 25% (w/w) of delphinidin-3-O-sambubioside-5-O-glucoside, from about 1 to about 50% (w/w) of delphinidin-3,5-O-diglucoside, from about 0.1% to about 20% (w/w) of cyanidin-3-O-sambubioside-5-O-glucoside, from about 0.05% to about 20% (w/w) of cyanidin-3,5-O-diglucoside, from about 0.015% to about 15% (w/w) of delphinidin-3-O-sambubioside, from about 0.1% to about 25% (w/w) of delphinidin-3-O-glucoside, from about 0.02% to about 15% (w/w) of cyanidin-3-O-sambubioside, or from about 0.05% to about 15% (w/w) of cyanidin-3-O-glucoside.

13. The method of claim 12, wherein said at least one anthocyanidin comprises: from about 0.1% to about 25% (w/w) of delphinidin-3-O-sambubioside-5-O-glucoside, from about 1 to about 50% (w/w) of delphinidin-3,5-O-diglucoside, from about 0.1% to about 20% (w/w) of cyanidin-3-O-sambubioside-5-O-glucoside, from about 0.05% to about 20% (w/w) of cyanidin-3,5-O-diglucoside, from about 0.015% to about 15% (w/w) of delphinidin-3-O-sambubioside, from about 0.1% to about 25% (w/w) of delphinidin-3-O-glucoside, from about 0.02% to about 15% (w/w) of cyanidin-3-O-sambubioside, and from about 0.05% to about 15% (w/w) of cyanidin-3-O-glucoside.

14. The method of claim 10, said anthocyanidin(s) administered in an amount of about 200 mg.

15. The method of claim 11, said anthocyanidin(s) administered in an amount of about 200 mg.
